(19) United States
(12) Patent Application Publication
(10) Pub. No.: US 2022/0409649 A1
(43) Pub. Date: Dec. 29, 2022

(54) a-GLUCOSIDASE INHIBITOR, INVERTASE INHIBITOR, AND SUGAR ABSORPTION INHIBITOR

(71) Applicants: Kinki University, Higashiosaka-shi, Osaka (JP); Maple Farms Japan, Inc., Osaka-shi, Osaka (JP)

(72) Inventors: Atsushi TAGA, Higashiosaka-shi, Osaka (JP); Tetsushi YAMAMOTO, Higashiosaka-shi, Osaka (JP); Kanta SATO, Higashiosaka-shi, Osaka (JP)

(21) Appl. No.: 17/633,869

(22) PCT Filed: Aug. 3, 2020
(86) PCT No.: PCT/JP2020/029719

§ 371 Date: Feb. 8, 2022

(30) Foreign Application Priority Data
Aug. 15, 2019 (JP) 2019-149053
Apr. 28, 2020 (JP) 2020-079837

(51) Int. Cl. A61K 31/7016; A61K 36/20; A23L 33/10

(52) U.S. Cl. CPC A61K 31/7016; A61K 36/20; A23L 33/10

(57) ABSTRACT

[Object] To provide a composition having an excellent α-glucosidase inhibitory effect or invertase inhibitory effect.

[Solution] A compound (I) contains a compound represented by a structural formula described below as an active ingredient,

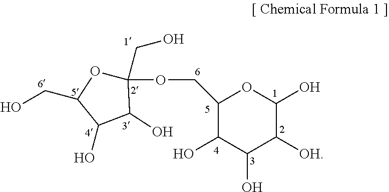 [SMILES: OCC1OC(CO)(OCC2OC(O)C(O)C(O)C2O)C(O)C1O]

### TECHNICAL FIELD

The present invention relates to an α-glucosidase inhibitor inhibiting an enzyme activity of α-glucosidase. The present invention also relates to an invertase inhibitor inhibiting an enzyme activity of invertase. The present invention also relates to a saccharide absorption inhibitor.

### BACKGROUND ART

α-glucosidase is a glycolytic enzyme localized on an epithelium of a small intestine and involved in glycoprotein processing and glycogenolysis. An α-glucosidase inhibitor specifically inhibiting α-glucosidase can directly inhibit saccharide absorption when ingested orally (Patent Literature 1).

Invertase is a digestive enzyme present in a wall of the small intestine and is an enzyme hydrolyzing sucrose. Sucrose ingested by a human and captured by the small intestine is hydrolyzed into glucose (grape sugar) and fructose (fruit sugar) with the invertase. The glucose and the fructose are absorbed from small intestinal epithelial cells into blood vessels and transported to organs in a body through the blood vessels. An invertase inhibitor can directly inhibit absorption of sucrose and other fructosyl saccharides when ingested orally (Patent Literature 2).

### CITATION LIST

### Patent Literature

Patent Literature 1: Japanese Patent Application Laid-Open No. 2012-51916

Patent Literature 2: Japanese Patent Application Laid-Open No. 2016-153399

### SUMMARY OF INVENTION

### Technical Problem

It is an object of the present invention to provide a substance having an excellent α-glucosidase inhibitory effect, invertase inhibitory effect, or saccharide absorption inhibitory effect.

### Solution to Problem

The present inventors have conducted intensive studies to solve the above-described problem. As a result, the present inventors have found that disaccharides obtained from sap of a tree belonging to a genus of Acer in a family of Aceraceae has an excellent α-glucosidase inhibitory effect, invertase inhibitory effect, or saccharide absorption inhibitory effect. Then, the present inventors have further conducted studies, and thus have completed the present invention.

More specifically, the present invention provides an α-glucosidase inhibitor or an invertase inhibitor described below.

An α-glucosidase inhibitor or an invertase inhibitor containing a compound represented by a structural formula described below as an active ingredient,

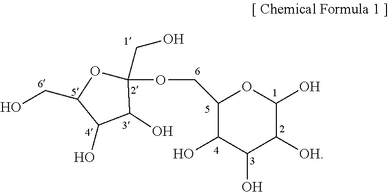 [SMILES: OCC1OC(CO)(OCC2OC(O)C(O)C(O)C2O)C(O)C1O]

The α-glucosidase inhibitor or the invertase inhibitor is obtained from sap of the tree belonging to the genus of Acer in the family of Aceraceae.

The tree belonging to the genus of Acer in the family of Aceraceae is at least one species selected from the group consisting of a sugar maple, a painted maple, a black maple, a red maple, a silver maple, a striped maple, a mountain maple, and a Norway maple.

The α-glucosidase inhibitor inhibits an enzyme activity of maltase.

The α-glucosidase inhibitor inhibits an enzyme activity of isomaltase.

The α-glucosidase inhibitor inhibits an enzyme activity of sucrase.

A food containing the α-glucosidase inhibitor or the invertase inhibitor.

The present invention also provides a saccharide absorption inhibitor containing a compound (I) represented by a structural formula described below,

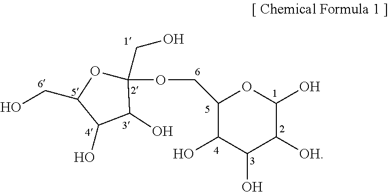 [SMILES: OCC1OC(CO)(OCC2OC(O)C(O)C(O)C2O)C(O)C1O]

The present invention also provides a saccharide composition containing the saccharide absorption inhibitor and sucrose.

The present invention also provides a food containing the saccharide absorption inhibitor.

Hereinafter, the present invention is described in detail.

The α-glucosidase inhibitor, the invertase inhibitor, or the saccharide absorption inhibitor according to the present invention is obtained from sap of a tree belonging to a genus of Acer in a family of Aceraceae. The tree belonging to the genus of Acer in the family of Aceraceae from which the sap is acquired is preferably one of a sugar maple, a painted maple, a black maple, a red maple, a silver maple, a striped maple, a mountain maple, and a Norway maple, and the sugar maple is more preferable. The sap of the sugar maple has particularly good quality and is easily available in large quantities among the sap of the tree belonging to the genus of Acer in the family of Aceraceae.

The sap has different component ratios, colors, scents, and the like depending on a season of collection from the tree, but can be used regardless of the season of collection. The sap may contain preservatives. Examples of the preservatives include 1,3-buthanediol, methyl 4-hydroxybenzoate, and the like. Maple syrup is produced by heating and concentrating the sap of the tree belonging to the genus of Acer in the family of Aceraceae about 40 times. Maple sugar is produced by completely removing moisture from the maple syrup.

The sap of the tree belonging to the genus of Acer in the family of Aceraceae is collected by known steps. More specifically, the sap is obtained by making a hole in the trunk of the tree belonging to the genus of Acer in the family of Aceraceae and collecting overflowing sap (hereinafter, sometimes referred to as “sap”, or “maple sap”). The maple syrup is a concentrate of the obtained sap. As a method for concentrating the sap, any appropriate method can be adopted. For example, the sap is concentrated by a heat concentration method, a non-heat concentration method (vacuum concentration, freeze concentration, membrane concentration, and the like) or a combination thereof.

The main ingredient of the maple syrup and the maple sugar is sucrose, and in addition thereto, the maple syrup and the maple sugar contain a few percent of glucose and trace amounts of monosaccharides and oligosaccharides. Major saccharides contained in the maple syrup and the maple sugar, i.e., glucose, fructose, and sucrose, can be analyzed by gas chromatography or anion exchange chromatography, for example. Reducing sugars contained in the maple syrup and the maple sugar can be analyzed by performing capillary electrophoresis after derivation with PMP (1-phenyl-3-methyl-5-pyrazolone). However, the PMP derivatization is not suitable for analyzing fructosyl saccharide having no reducing ends. Hence, rare saccharides and saccharides having no reducing ends contained in the maple syrup and the maple sugar have not been studied sufficiently.

Before the derivatization with PMP, the present inventors caused digestion of the fructosyl saccharide by invertase to remove fructose residues from the reducing ends. Thereafter, the present inventors analyzed the PMP-derivatives by capillary electrophoresis, and thus have found saccharides interacting with invertase, i.e., saccharides according to the present invention.

The saccharides interacting with invertase are obtained by ultra-filtrating the sap of the tree belonging to the genus of Acer in the family of Aceraceae at 10 kDa to remove proteins, gel-filtrating the resultant sap to obtain a further molecular weight fraction, and then purifying the fraction by high performance liquid chromatography (HPLC), for example. The fraction obtained by the HPLC was subjected to the capillary electrophoresis in the same manner as described above, and peaks of the purified saccharides were confirmed to coincide with peaks of the saccharides interacting with invertase.

The HPLC was performed after acid hydrolysis to analyze the composition of the purified saccharides. As a result, two major peaks corresponding to glucose and fructose were observed. Since the peak area of glucose and the peak area of fructose were almost the same, the purified saccharides were presumed to be disaccharides containing glucose and fructose. To confirm that the purified saccharides were hexose disaccharides containing glucose and fructose, the molecular weight was measured by LC-ESI-MS/MS. By analyzing the purified saccharides after the PMP derivatization, the observed mass was m/z 673.26 as [M+H]⁺ and the product ion was m/z 511.33, which coincided with the mass of the hexose saccharides after the PMP derivatization. Further, NMR analysis was performed to clarify the structure of the purified disaccharides. The obtained NMR signals of hydrogen (proton) and carbon (carbon) are shown in Table 1. These chemical shifts showed that the structure of the purified disaccharide (compound (I)) was as follows.

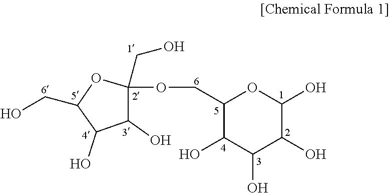 [SMILES: OCC1OC(CO)(OCC2OC(O)C(O)C(O)C2O)C(O)C1O]

The compound (I) described above is usable as it is as an α-glucosidase inhibitor, an invertase inhibitor, or a saccharide absorption inhibitor, and is also usable in the form of an extract or a powder by being concentrated or removing a solvent appropriately. Specifically, the compound (I) is useful as a therapeutic agent or a preventive agent for diabetes, obesity, and the like. The compound (I) may be prescribed to a human body or an animal as a composition with a pharmaceutically acceptable medium for injection, transrectal administration, parenteral administration, oral administration, and the like. The compound (I) described above may be added to foods for oral ingestion. Examples of the foods include beverages, confectioneries, cooked foods, seasonings, and the like. The compound (I) described above may be formed into a saccharide composition containing other saccharides, such as sucrose. Examples of the saccharide composition include sugars, sweeteners, maple syrup, maple sugar, and the like to which the compound (I) described above was added.

### Advantageous Effects of the Invention

The compound according to the present invention has an excellent α-glucosidase inhibitory action, invertase inhibitory action, or saccharide absorption inhibitory action.

### BRIEF DESCRIPTION OF DRAWINGS

FIG. 1 illustrates results obtained by performing PMP derivation with respect to maple syrup and performing capillary electrophoresis.

FIG. 2 illustrates results obtained by performing the PMP derivation with respect to invertase-digested maple syrup and performing the capillary electrophoresis.

FIG. 3 illustrates results obtained by performing the PMP derivation with respect to the invertase-digested maple syrup, adding invertase further, and performing the capillary electrophoresis.

FIG. 4 illustrates results obtained by ultra-filtrating maple syrup and performing HPLC.

FIG. 5 illustrates results obtained by performing the HPLC after performing acid-hydrolysis with respect to a fraction indicated by a * mark in FIG. 4 in the HPLC.

FIG. 6 illustrates screening results of inhibitory enzymes by a compound (I).

FIG. 7 illustrates changes with time of plasma glucose and insulin when normal rats were orally administered only with sucrose and when the normal rats were orally co-administered with sucrose and the compound (I).

FIG. 8 illustrates changes with time of plasma glucose and insulin when OLETF rats were orally administered only with sucrose and when the OLETF rats were orally co-administered with sucrose and the compound (I).

### DESCRIPTION OF EMBODIMENTS

### Examples

Hereinafter, the present invention is described in detail with reference to examples. It is a matter of course that the present invention is not limited to the examples described below.

[α-Glucosidase Inhibition]

Hereinafter, an α-glucosidase inhibitory effect was evaluated with respect to a compound (I).

[PMP Derivatization Processing of Sample]

The compound (I) was obtained from sap of trees belonging to a genus of Acer in a family of Aceraceae and maple syrup (BASCOM MAPLE FARMS INC.: hereinafter, also simply referred to as “sap and the like”). Specifically, 50 μL of 0.3 mol/L sodium hydroxide solution and 50 μL of 0.5 mol/L 1-phenyl-3-methyl-5-pyrazolone (hereinafter also referred to as “PMP”: manufactured by Kishida Chemical Co., Ltd.) methanolic solution were added to a dried sample of sap equivalent to 200 μL (10 μL of maple syrup or 10 mg of maple sugar), and the mixed liquid was heated at 70° C. for 30 minutes. The heated mixed liquid was neutralized by adding 50 μL of 0.3 mol/L hydrochloric acid, diluted with 100 μL of distilled water, and extraction was performed three times with 200 μL of chloroform to remove an excessive PMP reagent. By this process, PMP-derivatives for capillary electrophoresis were obtained.

### Purification of Compound (I)

The sap and the like were ultra-filtrated with a 10 kDa filter to remove proteins, and the obtained filtrate was gel-filtrated for performing further molecular weight fractionation. Gel filtration was performed using water as a mobile phase using Sephadex G-15 having a length of 1000 mm and an internal diameter of 28 mm, and fractions were collected by a fraction collector (Model 2110 manufactured by Bio-Rad Laboratories, Inc.). The obtained fractions were purified by high performance liquid chromatography (hereinafter, also referred to as “HPLC”). The peak corresponding to the compound (I) was observed between 32 and 33 minutes, and a fraction containing the compound (I) in high purity was collected with the peak as a criterion. The obtained solution was liophylized and used as a standard substance. 100 μg of the compound (I) was dissolved in 100 μL of water, and then the HPLC was performed.

[Capillary Electrophoresis]

Agilent 3D capillary electrophoresis system (Model G1600A manufactured by Waldbronn) equipped with a diode array UV detector was used. Samples were injected under a pressure of 50 mbar for 4 seconds. Separation was performed in a fused silica capillary column (manufactured by GL Science Inc., total length: 58.5 cm, effective length: 50 cm, internal diameter: 50 μm) having a non-treated inner surface. 200 mmol/L of a borate buffer solution for a background electrolyte (BGE) was obtained by adding pellets and 0.1 mol/L of a sodium hydroxide aqueous solution to a borate aqueous solution having the concentration slightly higher than 200 mmol/L, adjusting the pH to 10.5 using a pH meter, and adjusting the volume to 200 mmol/L using a volumetric flask. A voltage of 15 kV was applied to both ends of the capillary. Before injecting each sample, the capillary was conditioned by continuous rinsing with 0.5 mol/L sodium hydroxide for one minute and with the BGE for five minutes using a flush mode of the system. Detection was performed by monitoring UV absorption at 245 nm. Measurement was performed at 25±1° C.

[HPLC]

An HPLC system is constituted by a pump (Model LC-10AD manufactured by Shimadzu Corporation), a degasser (Model DGU-12A manufactured by Shimadzu Corporation), and a corona Veo detector (manufactured by Thermo Fisher Scientific, Inc.). Asahipak NH2P-50 4E column (5 μm, 4.6 mm internal diameter×250 mm, manufactured by Showa Denko K.K.) was used, and acetonitrile/water (3:1; v/v) was used as a mobile phase. Elution was performed at a flow rate of 1 ml/min at room temperature (about 23° C.). 20 μL of the sample was injected. In purification and fractionation, Asahipak NH2P-50 column (5 μm, 10.0 mm internal diameter×250 mm, manufactured by Showa Denko K.K.) was used and the flow rate was set to 2 mL/min. Using an adjustable splitter (manufactured by Thermo Fisher Scientific, Inc.) and setting the split ratio to 1:20, detection was performed at a low flow rate, and fractionation was performed at a high flow rate.

[Structural Analysis of Compound (I)]

LC-ESI-MS/MS analysis was performed using Finnigan LTQ linear ion trap mass spectrometer (manufactured by Thermo Fisher Scientific, Inc.) equipped with an ES ion source, Paradigm MS4 pump (manufactured by Michrom Bioresources Inc.), and an autosampler (HTCPAL, CTC Analytics). The conditions for ionization were as follows.

Ion source voltage: 4.5 kV

Capillary temperature: 275° C.

Capillary voltage: 25 V

Sheath gas (N2 gas): Flow rate of 50

Auxiliary gas (N2 gas): Flow rate of 5

Tube lens offset voltage: 90 V

Helium gas was used as a collision gas for Collision Induced Dissolution (CID) analysis. The normalized collision energy and the activation Q value were set to 35% and 0.18, respectively. As an LC column, TSK gel ODS-100S (manufactured by Tosoh Corporation, 5 μm, 150 mm×2.0 mm internal diameter) was used. ¹H and ¹³C-NMR were obtained using JNM-ECA800 instrument operating at 800 MHz and 200 MHz, respectively. NMR measurement samples were dissolved in heavy water.

[Invertase Digestion of Maple Syrup]

Enzyme reaction was performed by adding 40 μL of 5 mmol/L acetate buffer solution with the pH of 4.5 and 5 μL of 100 U/mL invertase to 10 mg of maple syrup, and incubating the mixture at 37° C. for 30 minutes. The reaction mixture was heated in a water bath for one minute to inactivate the enzyme. After evaporated and dried at room temperature, an invertase-digested substance was PMP-derivatized under the same conditions as those of an undigested sample. 50 μL of the PMP-derivatives were evaporated and dried, and then redissolved in 45 μL of 5 mmol/L acetate buffer solution with the pH of 4.5, and pre-incubation was performed for five minutes. Then, 5 μL of 100 U/mL invertase solution was added, and incubation was performed for 15 minutes. The reaction liquid was heated in a water bath for one minute to inactivate the enzyme, and analyzed by the capillary electrophoresis.

[Analysis of Invertase Inhibition by Compound (I)]

For invertase inhibition analysis, 100 μg of sucrose as a substrate and 1 μg, 10 μg, or 100 μg of the compound (I) being the invertase inhibitor were added to 50 μL of 100 mmol/L acetate buffer solution, and pre-incubation was performed at 37° C. for five minutes. Thereafter, 50 μL of 0.2 U/mL invertase solution was added, and incubation was performed for 15 minutes. Thereafter, 10 μL of the reaction mixture was heated in a water bath to inactivate the enzyme and evaporated and dried, and the PMP derivatization was performed. A substance subjected to the enzyme reaction under the same conditions without adding the inhibitor was used as a blank. In each sample, the concentration required for the compound (I) to inhibit 50% of the enzyme was defined as IC₅₀. An inhibition rate by the compound (I) was calculated using a following equation.

Inhibition rate (%)=[D1−(D2−D3)/1]×100 (Equation 1)

D1: Peak area of glucose in blank sample

D2: Peak area of glucose in each sample after enzyme reaction

D3: Peak area of glucose contained in inhibitor as an impurity

The IC₅₀ value was calculated from a dose-response curve of the compound (I) used as the inhibitor.

[Screening of Inhibition Analysis by Compound (I)]

As a crude enzyme mixture, 50 mg of rat intestinal acetone powder was added to 450 μL of 50 mmol/L phosphate buffer solution (pH 6.0), the mixture was stirred for 30 seconds, and then homogenized. Thereafter, the mixture was centrifuged (10000 rpm, 4° C., 20 minutes), and the supernatant was used as a purified enzyme (50 mg/450 μL). Sucrose and two types of glucose disaccharides (maltose and isomaltose) having different bonds were used as substrates. 3.4 mg of each of the two types of substrates was dissolved in 100 μL of phosphate buffer solution, and each solution was used as a substrate solution. A solution obtained by dissolving 3.4 mg of the compound (I) as a competitive inhibitor in 1 mL of phosphate buffer solution and diluting 100 times, was used as an inhibitor solution. 100 μL of the substrate solution and 10 μL of the inhibitor solution were mixed, and pre-incubation was performed for five minutes. Then, 90 μL of an enzyme solution was added to start incubation. Five hours later, 10 μL of the reaction solution was heated in a water bath for 10 minutes to stop the reaction, followed by the PMP derivatization, and then the capillary electrophoresis. An enzyme reaction solution under the same conditions without the inhibitor was used as a blank. The inhibition rate was calculated for each substrate using the same equation as that in the invertase inhibition analysis.

[Oral Glucose Tolerance Testing (OGT Testing) Using Sucrose]

Glucose tolerance testing of normal Wistar type rats and OLETF rats with diabetes was performed using the compound (I). After fasted for 14 hours, glucose was loaded, and blood taken from tail veins of the rats was subjected to various tests.

An aqueous solution containing 0.5 mg/ml of sucrose (hereinafter, also referred to as “A Liquid”) and an aqueous solution containing 0.5 mg/ml of sucrose and 0.085 mg/ml of the compound (I) (hereinafter, also referred to as “B Liquid”) were prepared. Six normal 7-week-old rats (male) were divided into two groups. In one group, the A liquid was orally administered such that an amount of sucrose was 1.5 g/kg based on the rat body weight. In the other group, the B liquid was orally administered such that the amount of sucrose was 1.5 g/kg based on the rat body weight. Blood was taken from the tail veins of the rats before the administration and in 30 minutes, 60 minutes, 90 minutes, and 120 minutes after the administration, and then plasma was obtained by centrifugation. For the obtained plasma, glucose and insulin were quantified.

[Evaluation]

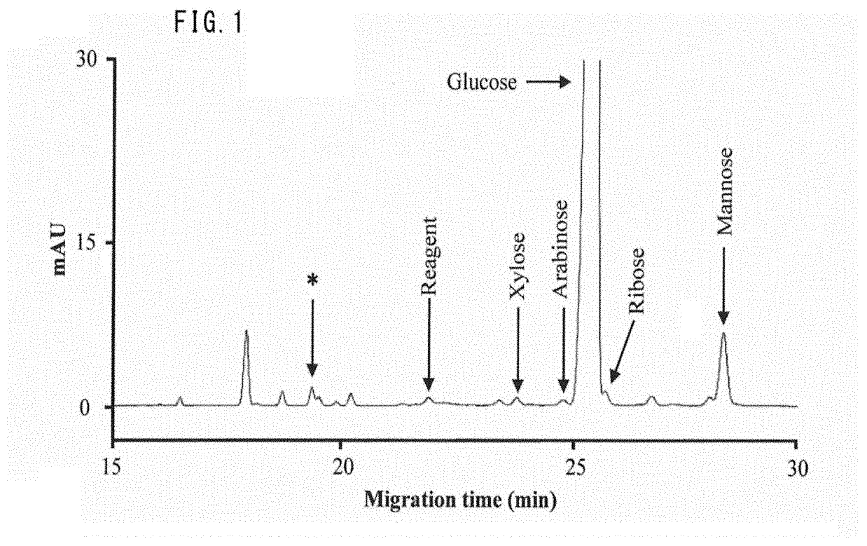

FIG. 1 illustrates results obtained by performing the PMP derivation with respect to the maple syrup and analyzing by the capillary electrophoresis. As a result, a plurality of peaks was detected, and each peak was identified as glucose, xylose, arabinose, mannose, and ribose. Further, the compound (I) indicated by a * mark was identified.

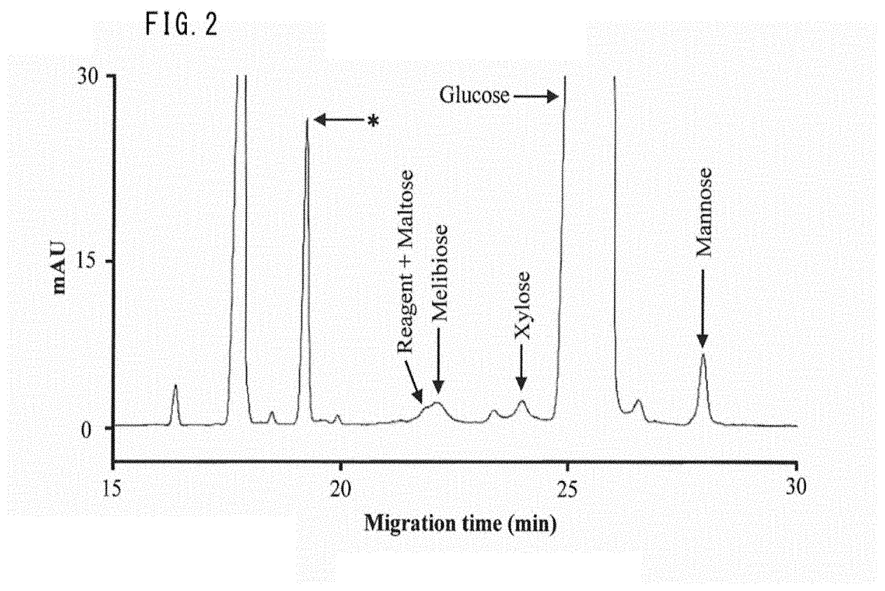

FIG. 2 illustrates results of the capillary electrophoresis with respect to the invertase-digested maple syrup. As a result of invertase-digesting the maple syrup, peak areas of glucose, xylose, and the compound (I) (* mark) increased as compared with those in the results obtained in the invertase-undigested maple syrup (FIG. 1).

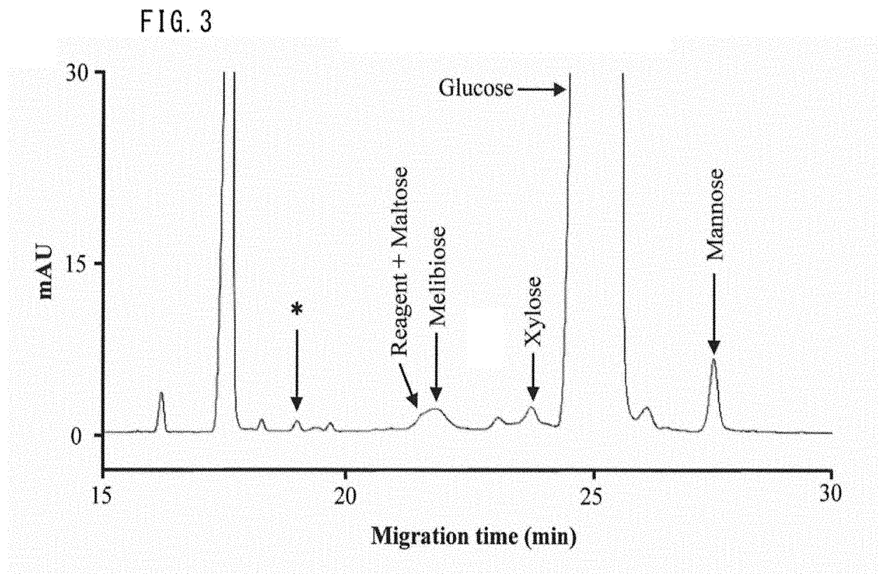

FIG. 3 illustrates results obtained by further adding invertase to the invertase-digested maple syrup and similarly analyzing by the capillary electrophoresis. When invertase was added to the invertase-digested maple syrup, the peak area of the compound (I) decreased, but significant changes were not observed in the peak areas of other saccharides. Based on this result, the compound (I) was presumed to interact with invertase.

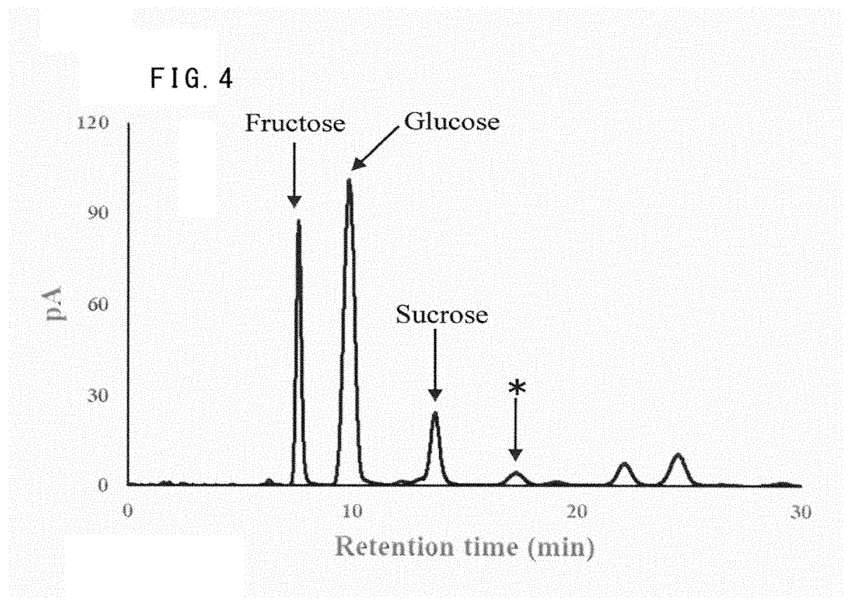

FIG. 4 illustrates results obtained by ultra-filtrating the maple syrup and analyzing by the HPLC. With respect to the results in FIG. 4, peaks of sucrose, fructose, and glucose were identified using standard substances. A fraction indicated by the peak (* mark) between 16 to 17 minutes was fractionated, PMP-derivatized, and then analyzed by the capillary electrophoresis. As a result, the substance contained in the peak between 16 to 17 minutes in the HPLC coincided with the peak of oligosaccharide interacting with invertase.

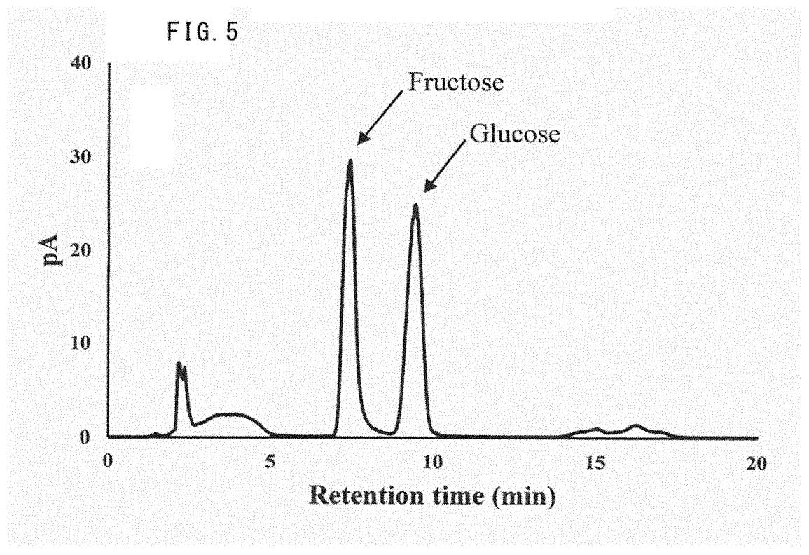

FIG. 5 illustrates results obtained by analyzing by the HPLC after performing acid-hydrolysis with respect to the fraction indicated by the * mark in the HPLC. Two major peaks illustrated in FIG. 5 were confirmed to correspond to fructose and glucose. Since the two peak areas were almost equal, the compound (I) was presumed to be disaccharide containing fructose and glucose.

As a result of analyzing the PMP-derivatives of the compound (I) by the LC-ESI-MS/MS and measuring the molecular weight, the mass was m/z 673.26 as [M+H]⁺, which coincided with the mass of PMP-derivatized disaccharide. The product ion was m/z 511.33, which coincided with the mass of PMP-derivatized monosaccharide.

Table 1 illustrates results of analyzing the compound (I) by the NMR. Based on this chemical shift, it was concluded that the compound (I) had a structure represented by a structural formula described below.

TABLE 1
Chemical shift
Oligosaccharide

| Residu | Position | δH | δC | J H, H | Type | Residu | Position | H (proton) | C (carbon13) | J (Hz) | Type |
|---|---|---|---|---|---|---|---|---|---|---|---|
| Glc α | 1 | 5.07 | 94.72 | 3.8 | d | Glc β | 1 | 4.49 | 98.57 | 8 | d |
| | 2 | 3.38 | 74.05 | 3.8, 9.8 | dd | | 2 | 3.09 | 76.67 | 8.0, 9.3 | dd |
| | 3 | 3.54 | 75.25 | — | m | | 3 | 3.33 | 78.23 | 9.3, 9.3 | dd |
| | 4 | 3.29 | 72.32 | 9.0, 9.0 | dd | | 4 | 3.29 | 72.25 | 9.0, 9.0 | dd |
| | 5 | 3.77 | 73.27 | 1.9, 9.2, 9.7 | ddd | | 5 | 3.40 | 77.58 | 2.1, 8.7, 9.7 | ddd |
| | 6 | 3.82 | 63.38 | 2.0, 11.0 | dd | | 6 | 3.87 | 63.38 | 2.1, 10.9 | dd |
| Fru β | 1′ | 3.60 | 62.75 | — | m | Fru β | 1′ | 3.54 | 62.72 | — | m |
| | 2′ | — | 106.31 | — | — | | 2′ | — | 106.34 | — | — |
| | 3′ | 4.03 | 79.42 | 8.5 | d | | 3′ | 4.02 | 79.52 | 8.5 | d |
| | 4′ | 3.97 | 77.06 | 8.1, 8.2 | dd | | 4′ | 3.96 | 77.21 | 8.0, 8.3 | dd |
| | 5′ | 3.72 | 83.75 | 7.4 | t | | 5′ | 3.72 | 83.81 | 7.4 | t |
| | 6′ | 3.54 | 64.98 | — | m | | 6′ | 3.66 | 65.10 | — | m |

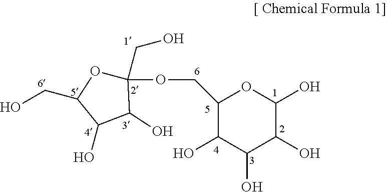 [SMILES: OCC1OC(CO)(OCC2OC(O)C(O)C(O)C2O)C(O)C1O]

Table 2 illustrates results of invertase inhibition analysis by the compound (I). The inhibition rates when 1 μg, 10 μg, and 100 μg of the compound (I) were added were 40.3%, 43.6%, and 65.2%, respectively, and a linearity (R²=0.99984) was observed between the concentration of the compound (I) and the inhibition rate. The IC₅₀ calculated using the straight line was 1.17 mmol/L.

TABLE 2
Table. 2. Inhibitory effects of maplehiose against glycolytic enzyme

| | inhibitor (μg) | Peak response (Glc) | Peak response (b-Glc) | Inhibition (%) | IC50 (mmol/1) |
|---|---|---|---|---|---|
| invertase | — | 30.5 | — | | |
| | 1 | 18.2 | N.D | 40.3 | 1.17 |
| | 10 | 17.2 | N.D | 43.6 | |
| | 100 | 23.1 | N.D | 65.2 | |
| Maltase | — | 44.3 | — | | |
| α-(1-4)glucosidase | 1 | 27 | N.D | 39.1 | 1.72 |
| | 10 | 22.4 | N.D | 49.4 | |
| | 100 | 20.3 | N.D | 54.2 | |

N.D = Not detectable

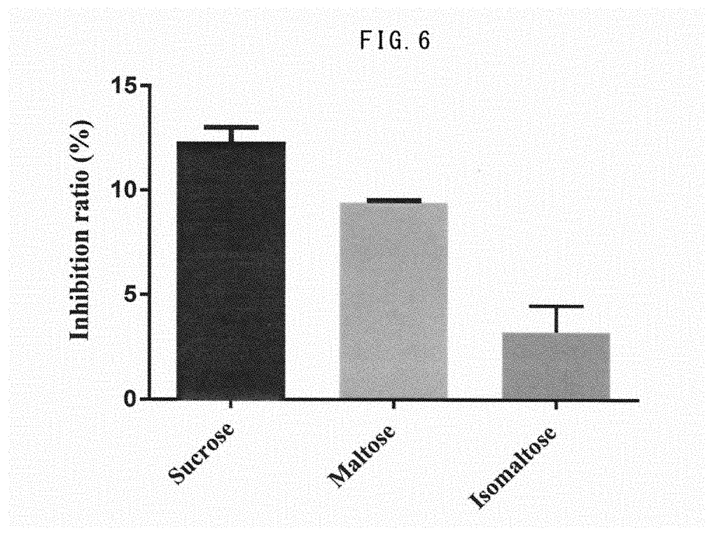

FIG. 6 illustrates screening results of an enzyme inhibitory activity by the compound (I). The inhibition rates by the compound (I) when sucrose, maltose, and isomaltose were used as the substrate were 12.3%, 9.4%, and 3.3%, respectively. Table 2 illustrates results of analyzing maltase inhibition by the compound (I). The inhibition rates when 1 μg, 10 μg, and 100 μg of the compound (I) were added were 39.1%, 49.4%, and 54.2%, respectively. The IC₅₀ calculated using a straight line calculated from the concentrations and the inhibition rates of the compound (I) was 1.72 mmol/L.

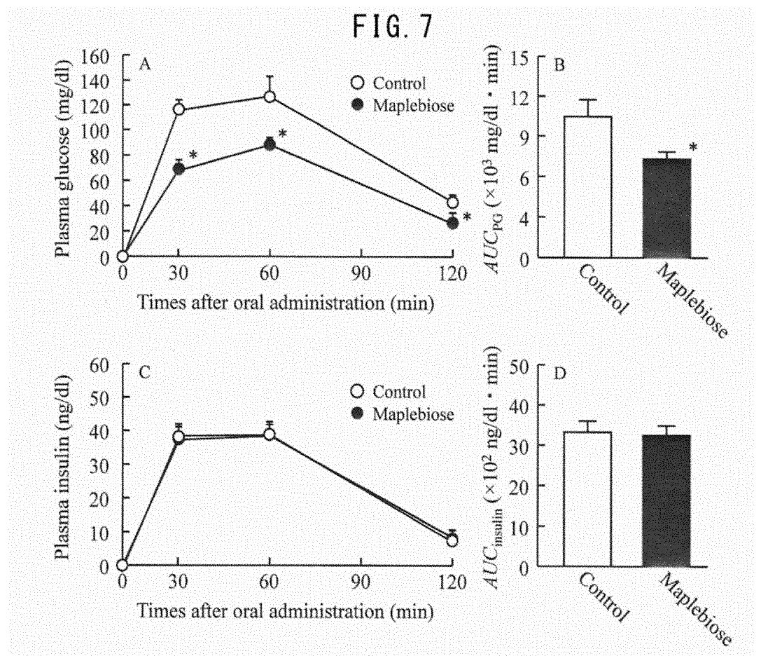

FIG. 7 illustrates changes in plasma glucose and insulin when normal rats were orally co-administered with sucrose and the compound (I). Changes with time of insulin were similar regardless of the presence or absence of the compound (I). However, the plasma glucose value was significantly lower in the rats administered with the compound (I) than in the rats not administered with the compound (I). In FIG. 7, the compound (I) is indicated as Maplebiose.

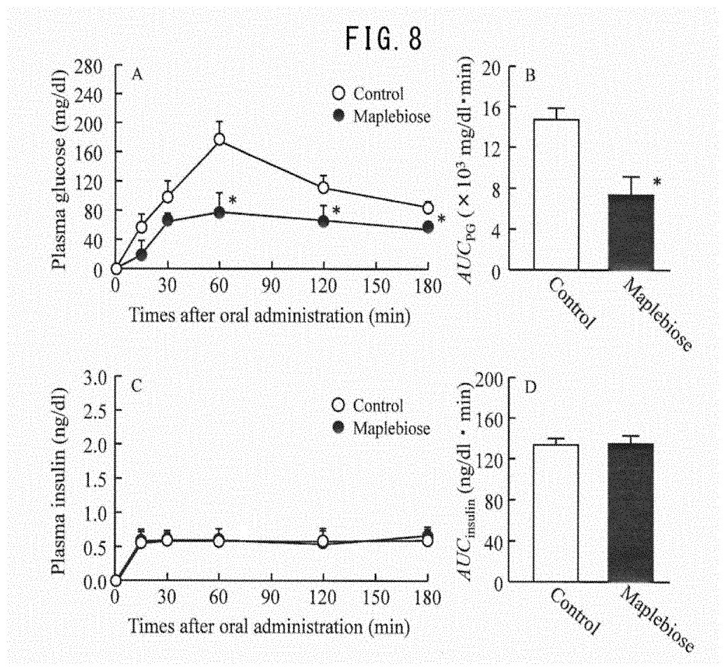

FIG. 8 illustrates changes in plasma glucose and insulin when OLETF rats with diabetes were orally co-administered with sucrose and the compound (I). Changes with time of insulin were similar regardless of the presence or absence of the compound (I). However, the plasma glucose value in the rats administered with the compound (I) decreased to about 50% of that in the rats not administered with the compound (I). In FIG. 8, the compound (I) is indicated as Maplebiose.

1. An α-glucosidase inhibitor comprising:
a compound (I) represented by a structural formula described below as an active ingredient,

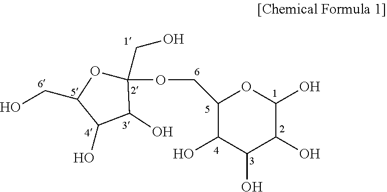 [SMILES: OCC1OC(CO)(OCC2OC(O)C(O)C(O)C2O)C(O)C1O]

2. The α-glucosidase inhibitor according to claim 1, which is obtained from sap of a tree belonging to a genus of Acer in a family of Aceraceae.

3. The α-glucosidase inhibitor according to claim 2, wherein
the tree belonging to the genus of Acer in the family of Aceraceae is at least one species selected from the group consisting of a sugar maple, a painted maple, a black maple, a red maple, a silver maple, a striped maple, a mountain maple, and a Norway maple.

4. The α-glucosidase inhibitor according to claim 1, which inhibits an enzyme activity of maltase.

5. The α-glucosidase inhibitor according to claim 1, which inhibits an enzyme activity of isomaltase.

6. The α-glucosidase inhibitor according to claim 1, which inhibits an enzyme activity of sucrase.

7. A food comprising:
the α-glucosidase inhibitor according to claim 1.

8. An invertase inhibitor comprising:
a compound (I) represented by a structural formula described below as an active ingredient,

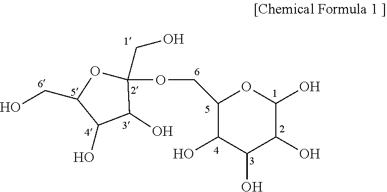 [SMILES: OCC1OC(CO)(OCC2OC(O)C(O)C(O)C2O)C(O)C1O]

9. The invertase inhibitor according to claim 8, which is obtained from sap of a tree belonging to a genus of Acer in a family of Aceraceae.

10. The invertase inhibitor according to claim 9, wherein
the tree belonging to the genus of Acer in the family of Aceraceae is at least one species selected from the group consisting of a sugar maple, a painted maple, a black maple, a red maple, a silver maple, a striped maple, a mountain maple, and a Norway maple.

11. A food comprising:
the invertase inhibitor according to claim 8.

12. A saccharide absorption inhibitor comprising:
a compound (I) represented by a structural formula described below,

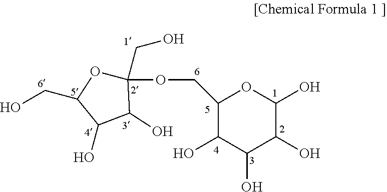 [SMILES: OCC1OC(CO)(OCC2OC(O)C(O)C(O)C2O)C(O)C1O]

13. A saccharide composition comprising:
the saccharide absorption inhibitor according to claim 12; and
sucrose.

14. A food comprising:
the saccharide absorption inhibitor according to claim 12.
